(19) United States
(12) Patent Application Publication
(10) Pub. No.: US 2023/0256437 A1
(43) Pub. Date: Aug. 17, 2023

(54) METHODS AND DEVICES FOR THE RAPID DETECTION OF SARS-COV-2/COVID-19 DISEASE

(71) Applicant: THE UNIVERSITY OF MASSACHUSETTS, Boston, MA (US)

(72) Inventors: Gulden Camci-Unal, Lowell, MA (US); Darlin Lantigua, Lawrence, MA (US)

(21) Appl. No.: 18/005,155

(22) PCT Filed: Jul. 15, 2021
(86) PCT No.: PCT/US2021/041723

§ 371 Date: Jan. 11, 2023

Related U.S. Application Data
(60) Provisional application No. 63/052,276, filed on Jul. 15, 2020.

(51) Int. Cl. B01L 3/00; G01N 33/543; G01N 33/569

(52) U.S. Cl. CPC B01L 3/502753; G01N 33/54391; G01N 33/56983; B01L 2300/087; G01N 2333/165

(57) ABSTRACT

Multilayer diagnostic microfluidic devices for the capture of a present or past SARS-CoV-2 infection and/or COVID-19 disease from a fluid sample using a multiplexed ELISA-based assay and their methods of use are described herein. The devices comprise a sample application layer comprising a sample application zone: a conjugation layer comprising a conjugation reagent in a conjugation zone, wherein the conjugation reagent comprises a detectable marker; a capture layer comprising a capture reagent in a capture zone; and an absorbent layer. The layers above tire capture layer are removable to expose a detectable signal in the capture zone. Detectable analytes include a SARS-CoV-2 protein or an antigenic fragment thereof, an IgM, IgG, or IgA antibody associated with a SARS-CoV-2 infection and or COVID-19 disease, or an inflammatory biomarker associated with a SARS-CoV-2 infection and/or COVID-19 disease.

### CROSS-REFERENCE TO RELATED APPLICATIONS

This application claims priority to U.S. Provisional Application 63/052,276 filed on Jul. 15, 2020, which is incorporated herein by reference in its entirety.

### FIELD OF THE DISCLOSURE

The present disclosure is related to methods and enzyme-linked immunosorbent assay (ELISA)-based assay devices for the rapid detection of biomolecules, such as antigens, antibodies, and inflammatory factors, associated with present and past SARS-CoV-2 infection and/or the COVID-19 disease.

### BACKGROUND

In mid-December 2019, an outbreak of a new kind of viral pneumonia was reported in Wuhan Province. China. The cause of these infections was identified as a novel coronavirus, close in phylogeny to the severe acute respiratory syndrome (SARS) virus of 2003 and Middle East respiratory syndrome (MERS) virus of 2012. The new virus has been named SARS-CoV-2, and the clinical presentation of SARS-CoV-2 infection has been named COVID-19.

Shortly after its emergence. SARS-CoV-2 virus spread across the globe, presenting a serious global public-health emergency. The World Health Organization (WHO) declared a Public Health Emergency of International Concern (PHEIC) on Jan. 31, 2020 and declared a global pandemic on Mar. 11, 2020. The estimated mortality rate is around 3 percent. See Huang, C. et al. Clinical features of patients infected with 2019 novel coronavirus in Wuhan, China. Lancet 395. 497 --- 506 (2020). The COVID-19 pandemic has caused substantial health impacts-as of Jul. 14, 2020, Johns Hopkins University counts 13.1 million worldwide confirmed COVID-19 diagnoses and 574,000 deaths. See Johns Hopkins University. Coronavirus Resource Center. The United States has suffered disproportionately, representing about 25 percent the world’s COVID-19 diagnoses and deaths. The true number of COVID-19-related deaths may be as much as double official counts. See, e.g., Thomas DM, Excess registered deaths in England and Wales during the COVID-19 pandemic, arXiv, last revised 2 Jun. 2020.

Efforts to curb the spread of SARS-CoV-2 virus have resulted in major disruptions in regular life activities, including strict travel restrictions and public accommodations closures. One important reason for these disruptions is significant ongoing uncertainty concerning person-to-person spread of the virus: we still generally do not know which individuals are or were infected. Among other complications, it has become apparent that a substantial proportion of SARS-CoV-2 carriers are asymptomatic and would have no reason to believe they may be infected. See, e.g., Nishiura H & Kobayashi ‘T’, Estimation of the asymptomatic ratio of novel coronavirus infections (COVID-19), Int J Infect Dis. 2020 May; 94: 154-155. Without convenient testing, most asymptomatic carriers will probably never know they were infected. See, e.g., Nishiura H et al., The Rate of Under Ascertainment of Novel Coronavirus Infection: Estimation Using Japanese Passengers Data on Evacuation Flights, J Clin Med. 2020 Feb; 9(2): 419. An effective, sensible pandemic containment strategy requires careful monitoring that identifies where the SARS-CoV-2 virus is, where it has been, and where it is going.

Unfortunately, the prevailing diagnostic method for current SARS-CoV-2 infection comprises a real-time reverse transcription polymerase chain reaction (real-time RT-PCR), which is expensive, time-consuming, and requires specialized training and laboratory equipment, and results generally take several days to produce. See, e.g., LabCorp® Product Information 139900 2019 Novel Coronavirus (COVID-19), NAA Test Kit (describing the expected turnaround time as 2-4 days). Accordingly, there remains a paramount unmet need for a rapid, effective, low-cost, easy-to-use test for current and past SARS-CoV-2 virus infection.

### BRIEF SUMMARY

In an aspect, a multilayer microfluidic device for singleplex and multiplex detection of a present or past SARS-CoV-2 infection and/or COVID-19 disease comprises
a sample application layer comprising a sample application zone,
a first side of a conjugation layer comprising a conjugation reagent in a conjugation zone, wherein the conjugation reagent comprises a detectable marker,
a first side of a capture layer comprising a capture reagent in a capture zone,
an absorbent layer,
wherein the sample application layer is in communication with a first side of the conjugation layer, a second side of the conjugation layer is in communication with a first side of the capture layer, and the second side of the capture layer is in communication with a first side of the absorbent layer,
wherein the sample application layer, the conjugation layer, the capture layer, and the absorbent layer each comprise porous media,
wherein the sample application zone, conjugation zone, and the capture zone are in communication such that an analyte in the sample application zone binds to the conjugation reagent in the conjugation reagent zone to form an analyte-conjugation reagent complex, and the analyte-conjugation reagent complex binds to the capture reagent in the capture zone to form a detectable signal in the capture zone,
wherein the sample application layer and the conjugation layer are above the capture layer and are removable to expose the detectable signal in the capture zone, and
wherein the analyte is a SARS-CoV-2 protein or an antigenic fragment thereof, an IgM, IgG, or IgA antibody associated with a SARS-CoV-2 infection and or COVID-19 disease, or an inflammatory biomarker associated with a SARS-CoV-2 infection and/or COVID-19 disease, and
wherein the detectable signal in the capture zone indicates a present or past SARS-CoV-2 infection and/or COVID-19 disease.

In another aspect, a method of detecting present or past SARS-CoV-2 infection, SARS-CoV-2 virus, and/or COVID-19 disease in a sample comprises
applying the sample to the sample application zone of the microfluidic device described herein,
optionally applying a buffer to the test sample to initiate flow and wash the layers, and
allowing the sample to flow from the sample layer, to the conjugation layer, to the capture layer, and
removing the layers that are on top of the capture layer to expose the capture zone of the capture layer,
wherein the presence of a detectable signal from the patient sample in the capture zone confirms a present or past SARS-CoV-2 infection, SARS-CoV-2 virus, and/or COVID-19 disease.

### BRIEF DESCRIPTION OF THE DRAWINGS

FIGS. 1A, 1B, 1C, 1D, 1E depict schematics of the layers comprising a multiplexed embodiment of a rapid SARS-CoV-2 detection assay device. Particularly, FIG. 1A depicts a schematic of a sample layer; FIG. 1B depicts a schematic of a blood separator membrane; FIG. 1C depicts a schematic of a conjugation layer having four conjugation zones, each conjugation zone comprising a capture reagent for a SARS-CoV-2/COVID-19 analytes, the capture reagent comprising a detectable marker; FIG. 1D depicts a schematic of a capture layer having four capture zones, each capture zone comprising a capture regent for the antigen-conjugation reagent complex; and FIG. 1E depicts a schematic of an absorption layer.

FIGS. 2A, 2B, 2C, 2D, 2E depict photographic images of the layers comprising a singleplexed embodiment of a rapid SARS-CoV-2 detection assay device with an analyte detection zone and a control zone. Particularly. FIG. 2A depicts a photograph of a sample layer; FIG. 2B depicts a photograph of a blood separator membrane; FIG. 2C depicts a photograph of a conjugation layer having two conjugation zones; FIG. 2D depicts a photograph of a capture layer having two capture zones; and FIG. 2E depicts a photograph of an absorption layer.

FIGS. 3A, 3B depict exploded views of exemplary layers of a rapid SARS-CoV-2 detection assay device. FIG. 3A depicts an exploded view of a multiplex embodiment of a rapid SARS-CoV-2/COVID-19 detection assay device. FIG. 3B depicts an exploded view of a singleplexed embodiment of a rapid SARS-CoV-2 detection assay device.

FIGS. 4A, 4B depict photographic images of a standard 1.5 mL Eppendorf® tube containing gold-nanoparticle-conjugated anti-SARS-CoV-2 nucleocapsid (N) and spike (S) protein antibodies.

FIGS. 5A, 5B, 5C illustrate a method of the paper-based point-of-care (POC) platform for COVID-19 diagnostics. FIG. 5A is a schematic representation of the fabrication of 5-layer paper-based POC devices for singleplex and multiplex detection using a wax printing method, FIG. 5B outlines the steps for sample testing in the diagnostic platform, and FIG. 5C illustrates the immunoassay approaches used for the detection of SARS-CoV-2 antigenic proteins (NP and SP) in the singleplex platform (8), SARS-CoV-2-SP IgG/IgM antibodies detection (9) and positive control development (10) during multiplex detection, interpretation of the colorimetric results formed in the device test readout layer (11).

FIGS. 6A, 6B, 6C, 6D, 6E, 6F, 6G, 6H, 6I, and 6J show the colorimetric detection and quantification of SARS-CoV-2 antigenic proteins (SP and NP) and COVID-19 antibodies (IgG and IgM). FIGS. 6A, 6B, 6C, 6D, 6E, and 6F depict images of the colorimetric results for the detection of individual SARS-CoV-2 proteins (NP and SP) during antigen testing in the singleplex diagnostic platform for SARS-CoV-2 detection. FIGS. 6G and 6H are graphs of color intensity (arbitrary units) versus test sample, quantifying the colorimetric results of antigen testing. The antigenic spike protein and nucleocapsid protein were individually detected in human blood at 100 µg/mL, 100 ng/mL, and 100 pg/mL concentrations. The test zone and control zones were labeled T and C, respectively. 15 µL of whole human blood in the presence of the viral proteins were detected in the test zone, T. The detection time was 5 minutes. The red color formation in the T zone decreased as a result of decreasing viral protein concentrations in the tested samples, as expected for a sandwich immunoassay. FIG. 6I is an illustration of the method steps used in the multiplex diagnostic platform. FIG. 6J is a graph of color intensity (arbitrary units) versus test sample, quantifying the colorimetric results of IgG/IgM antibody and SARS-CoV-2 SP viral subunit detection. Detection and quantification for multiplex detection of the SARS-CoV-2 SP viral subunit and IgG/IgM antibodies against SARS-CoV-2-S(P) within one diagnostic device. The test zones of the device targeting each specific analyte were labeled as IgG, IgM, S(P) for detection of the spike viral protein, and C for positive control. A sample spiked with each targeted analyte in human blood at a 100 µg/mL concentration. NIH ImageJ software was used for quantification of red color formation for all concentrations tested using these diagnostic platforms. This was accomplished by measuring the grey intensity in the images taken using an Android cellphone. Three replicates for each condition were performed for all concentrations. Statistical analyses were performed using GraphPad Prism (La Jolla, CA. USA). Error bars: ± SD, *p < 0.05, **p < 0.01, ***p < 0.001, and ****p < 0.0001.

FIGS. 7A, 7B, 7C, 7D, 7E, 7F, and 7G show the analytical specificity for cross-reactivity testing of pathogenic viruses and endogenous interferents. FIGS. 7A, 7B, 7C, 7D, and 7E are photographic images depicting the colorimetric results for the detection of pathogenic viruses (SARS-CoV, MERS-CoV, HIV, and Influenza A) spiked in human blood and tested in the singleplex platform with samples in the absence of SARS-CoV-2. FIG. 7F is a graph of quantification of the results of color formation observed in the test zone of the diagnostic devices challenged with each of the pathogenic virus tested in FIGS. 7A, 7B, 7C, 7D, and 7E. Each pathogenic virus was tested in triplicate, and results were quantified using NIH ImageJ software. The pathogenic viruses were tested at a 100 ng/mL concentration, and results were assessed by statistical analysis and compared to the negative control (0 ng/mL). Statistical analysis demonstrated minimal cross reactivity for the HIV virus and apparent differentiation between coronaviruses. FIG. 7G is a graph illustrating the interference test results in the presence of endogenous substances (bilirubin, cholesterol, uric acid, albumin, and gamma globulin) for the detection of the SARS-CoV-2 SP in the singleplex platform. The image labeled “SARS-CoV-2 + Endo. S” shows the colorimetric results for a cocktail containing 10 ng/mL SARS-CoV-2 SP viral antigen, 0.2 mg/mL uric acid, 120 mg/mL albumin, 120 mg/mL gamma globulin, 0.6 mg/mL bilirubin, and 5 mg/mL cholesterol in unmodified human blood. The quantified results of the tested cocktail showed no significant difference when compared to devices tested with samples in the presence of only SARS-CoV-2 SP.

FIGS. 8A, 8B, 8C, 8D, and 8E show the results of SARS-CoV-2 detection performance in different bodily fluids using the POC diagnostic platform. FIGS. 8A, 8B, 8C, and 8D depict photographic images of colorimetric results for the detection of the SARS-CoV-2 antigenic SP in samples such as saliva, nasal fluid, human blood, and urine. The SARS-CoV-2 SP viral protein was spiked in each specimen at a concentration of 100 ng/mL and tested in single devices as triplicates. FIG. 8E is a graph illustrating quantification of the colorimetric results obtained from the images taken with an Android cellphone camera. The colorimetric results were quantified using NIH ImageJ software, and results were evaluated through statistical analysis using GraphPad Prism (La Jolla, CA, USA). Error bars: ± SD, *p < 0.05. **p < 0.01, and ****p < 0.0001. The paper-based microfluidic platform successfully detected the antigenic proteins of SARS-CoV-2 in blood, saliva, nasal fluid, and urine. The saliva specimen provided the highest signal intensity (sensitivity) among all tested specimens. A statistical difference was observed in the detection signal among all specimen types, except between blood and nasal fluid, where no significance difference was observed.

The above-described and other features will be appreciated and understood by those skilled in the art from the following detailed description, drawings, and appended claims.

### DETAILED DESCRIPTION

The present disclosure provides ELISA-based assay devices for the rapid detection of biomolecules, such as antigens, antibodies, and inflammatory factors, and fragments thereof associated with present and past SARS-CoV-2 infection and/or COVID-19 disease. The test devices are safe, affordable, disposable, efficacious, reliable, easy-to-use, and do not require expensive or specialized laboratory equipment to operate, nor do the devices require specialized training to use. Advantageously, the devices can be multiplexed to detect multiple analytes. In addition, multiple specimen types can be used without sample pretreatment prior to testing.

The SARS-CoV-2 human pathogen belongs to a β-coronavirus group, and is characterized as having an enveloped, positive-sense single-strand RNA genome approximately 29.9 kbp in length. See Li, X., et al.. Molecular immune pathogenesis and diagnosis of COVID-19. J Pharm Analysis, 2020. Its genome is typical of known coronaviruses and was annotated as having at least 10 open reading frames (ORFs), encoding 27 proteins including four major structural proteins: spike (S), envelope (E), membrane protein, and nucleocapsid protein (N or NP). See Li, supra; Wu A., et al., Genome Composition and Divergence of the Novel Coronavirus Originating In China, Cell Host & Microbe, 2020. 27: pp. 325-328. The N protein and S protein have been identified as strong candidates for immunohistochemistry applications. See Fung, J., S.K.P. Lau, and P.C.Y. Woo, Antigen Capture Enzyme-Linked Immunosorbent Assay for Detecting Middle East Respiratory Syndrome Coronavirus in Humans, Methods Mol Biol. 2020;2099:89-97; Freeman B, et al., Validation of a SARS-CoV-2 spike protein ELISA for use in contact investigations and sero-surveillance, COVID-19 SARS-CoV-2 preprints from medRxiv and bioRxiv, https://doi.org/10.1101/2020.04.24.057323.

The SARS-CoV-2 nucleocapsid protein (N or NP) comprises a multifunctional structural protein, which forms helical ribonucleocapsid complexes, interacts with viral membrane proteins, and plays an important role in enhancing the efficiency of virus replication, transcription, and assembly. The N protein is a strong candidate for an immunohistochemistry-based detection assay because it is highly immunogenic and highly expressed. See Fung, J., S.K.P. Lau, and P.C.Y. Woo. Antigen Capture Enzyme-Linked Immunosorbent Assay for Detecting Middle East Respiratory Syndrome Coronavirus in Humans, Methods Mol Biol. 2020;2099:89-97.

The spike (S) protein is a homotrimeric glycoprotein, with each of the three spike monomers comprising two subunits. S1 and S2. expressed on the surface of the viral envelope to bind to host cellular receptors. Specifically, spike protein binds with angiotensin-converting enzyme 2 (ACE2) receptor, releasing its S1 subunits and ultimately leading to fusion of the viral and cellular membranes for the SARS-CoV-2 virus to achieve entry into the cell. See Lan J, et al.. Structure of the SARS-CoV-2 spike receptor-binding domain bound to the ACE2 receptor, Nature 581, 215-220 (2020). Spike protein is well-conserved, has a well-characterized receptor-binding domain (RBD), and is another strong candidate for an immunochemistry-based detection assay.

Provided this knowledge about SARS-CoV-2 morphology and genome, the present disclosure provides ELISA-based assay devices for the rapid detection of biomolecules, such as antigens, antibodies, and inflammatory factors, and fragments thereof associated with present and past SARS-CoV-2 infection.

The rapid detection assay device of the present disclosure adapts principles of enzyme-linked immunosorbent assays (ELISA), also sometimes called enzyme immunoassays (EIA), and applies these principles to an easy-to-use microfluidic test device. ELISA is a highly sensitive class of techniques that use immunohistochemistry for the detection of the presence or absence of a target analyte.

The most widely used ELISA format is the sandwich format. In a sandwich ELISA, the target analyte is bound to a conjugation reagent (e.g.. an antibody) that is specific for the target analyte. A capture reagent (e.g., an antibody) specific for the analyte-capture reagent complex then binds the analyte-conjugation reagent complex. Generally, to avoid cross-reactivity the conjugation antibody and capture antibody will be from different species, e.g.. rabbit IgG and goat IgG. Binding of the target analyte-conjugation antibody complex by the capture antibody provides a detectable signal that indicates the presence of the target antigen.

Microfluidic ELISA-based assays alleviate shortcomings of conventional plate-based ELISA. Microfluidics refers to a class of technologies that uses and/or controls the flow of microliter or smaller volume-scale of fluids. In microfluidic ELISA-based assays, such as home pregnancy test kits, a solution suspected of containing a target analyte is introduced into the microfluidic device. As the solution diffuses through porous media, the solution passes sequentially through a series of reagents pre-impregnated on the test media, i.e.. a capture reagent and a detection reagent.

In an aspect, a multilayer microfluidic device for the detection of a present or past SARS-CoV-2 infection and/or COVID-19 disease comprises
a sample application layer comprising a sample application zone,
a first side of a conjugation layer comprising a conjugation reagent in a conjugation zone, wherein the conjugation reagent comprises a detectable marker,
a first side of a capture layer comprising a capture reagent in a capture zone, and
a first side of an absorbent layer,
wherein the sample application layer is in communication with a first side of the conjugation layer, a second side of the conjugation layer is in communication with a first side of the capture layer, and the second side of the capture layer is in communication with a first side of the absorbent layer,
wherein the sample application layer, the conjugation layer, the capture layer, and the absorbent layer each comprise porous media,
wherein the sample application zone, conjugation zone, and the capture zone are in communication such that an analyte in the sample application zone binds to the conjugation reagent in the conjugation reagent zone to form an analyte-conjugation reagent complex, and the analyte-conjugation reagent complex binds to the capture reagent in the capture zone to form a detectable signal in the capture zone,
wherein the layers above the capture layer are removable to expose the detectable signal in the capture zone, and
wherein the analyte is a SARS-CoV-2 protein or an antigenic fragment thereof, an IgM, IgG, or IgA antibody associated with a SARS-CoV-2 infection and or COVID-19 disease, or an inflammatory biomarker associated with a SARS-CoV-2 infection and/or COVID-19 disease, and
wherein the detectable signal in the capture zone indicates a present or past SARS-CoV-2 infection and/or COVID-19 disease. Exemplary devices include vertical flow devices.

In an aspect, the device further comprises a sample treatment layer disposed on a side of the sample application layer opposite the conjugation layer, wherein the sample treatment layer comprises porous media. In an aspect, the sample treatment layer filters the sample as it enters the device. In another aspect, the sample treatment layer separates plasma from whole blood.

In an aspect, the porous media for the layers comprises paper such as Whatman® filter paper, blotting paper, cellulose acetate, nitrocellulose, fabric, cotton, nylon, or plastic, any products thereof, or a combination thereof.

In an aspect, the sample application layer, the conjugation layer and the capture layer each comprise a hydrophobic material that defines the boundary of the sample application zone, the conjugation zone, and the capture zone, respectively. Exemplary hydrophobic materials include waxes, which can be applied using a wax printer. Hydrophobic pens can also be used to print the various zones.

In an aspect, the device comprises one or more adhesive layers disposed between the sample layer and the conjugation layer and/or between the conjugation layer and the capture layer. Exemplary adhesive layers include double-sided tapes, which can be patterned with a scalpel blade, laser-cutting device, or another automated cutting device.

A particular advantage of the device and methods described herein is the ability to provide multiplexed detection. Multiple SARS-CoV-2 protein/peptide antigens, as well as IgG, IgM, IgA, and inflammatory markers associated with viral infection such as SARS-CoV-2 infection or COVID-19 disease may be detected on a single device.

Exemplary SARS-CoV-2 protein/peptide antigens include a subunit, isoform, or antigenic peptide of SARS-CoV-2 Spike (S) protein, SARS-CoV-2 Membrane (M) protein, SARS-CoV-2 Nucleocapsid (N) protein, or SARS-CoV-2 Envelope (E) protein, or a combination thereof.

Exemplary IgG, IgM, and IgA antibodies for detection include human anti-SARS-CoV-2 (S) IgG antibody, human anti-SARS-CoV-2 (N) IgG antibody, human anti-SARS-CoV-2 (S) IgM antibody, human anti-SARS-CoV-2 (N) IgM antibody, human anti-SARS-CoV-2 IgA antibodies. The IgG can be IgG₁, IgG₂. IgG₃, IgG₄ or a combination thereof.

Exemplary inflammatory markers associated with SARS-CoV-2 infection or COVID-19 disease include interleukin-6 (IL-6), C-reactive protein (CRP), serum ferritin, and procalcitonin (PCT). The inflammatory markers are not limited to these and can be any inflammatory marker associated with a SARS-CoV-2 infection.

In specific aspect, the device comprises between one and ten sample application zones, between two and ten conjugation zones, and between two and ten capture zones. Each conjugation and capture zone can comprise the same or a different conjugation reagent and/or capture reagent.

The device can detect picogram to microgram amounts of the analyte in the sample, specifically picogram to nanogram amounts of analyte. In an aspect, the device detects less than or equal to 1,000 ng/ml, or less than or equal to 10 ng/ml, or less than or equal to 1 ng/ml, or less than or equal to 100 pg/ml, or less than or equal to 50 pg/ml of the analyte in the sample. In addition, only a small volume of sample is needed to detect the analyte. In an aspect, the volume of the sample is less than or equal to 1000 µl, or less than or equal to 100 µl, or less than or equal to 50 µl, or less than or equal to 20 µl, or less than or equal to 15 µl, or less than or equal to 10 µl.

The device comprises a control zone including a negative control, a positive control, or a combination thereof. As a negative control, the capture layer or detection layer has no capture antibody against the analyte-gold nanoparticle antibody complex. As a positive control, the capture layer will include an antibody against the metallic nanoparticle (e.g., gold nanoparticle)-labeled antibody.

The rapid detection assay device of the present disclosure adapts principles of enzyme-linked immunosorbent assays (ELISA). In practice, a sample suspected of containing an antigen associated with a present or past SARS-CoV-2 infection and/or COVID-19 disease is loaded onto the sample application layer. The sample then passes through the sample layer to the conjugation layer where the sample interacts with the conjugation reagent. If the sample contains the antigen, it binds to the conjugation reagent to form an analyte-conjugation reagent complex.

Exemplary conjugation reagents include antibodies specific for the target antigen. The conjugation reagent comprises a detectable marker such as a metallic nanoparticle to provide a detectable signal. Exemplary metallic nanoparticles include gold nanoparticles, palladium nanoparticles, platinum nanoparticles, silver nanoparticles, titanium nanoparticles, cerium oxide nanoparticles, and manganese oxide nanoparticles, specifically gold nanoparticles. The average particle size of the metallic nanoparticle provides optimal colorimetric results at high intensities visible to the naked eye and enhances the detection sensitivity of the assay platform. In an aspect, the metallic nanoparticle has a particle size of 0.1 to 200 nanometers, or 1 to 100 nm, or 5 to 50 nm, or 10 to 30 nm, or 10 to 25 nm. In an aspect, the metallic nanoparticle has a diameter of 20 nm. An “average particle size” or “average particle diameter” refer to a particle diameter corresponding to 50% of the particles in a distribution curve in which particles are accumulated in the order of particle diameter from the smallest particle to the largest particle and a total number of accumulated particles is 100%. Average particle size may be measured by methods such as, for example, a particle size analyzer, a transmission electron microscope (TEM) image, a scanning electron microscope (SEM) image, or by dynamic light scattering.

The analyte-conjugation reagent complex then passes through to the capture layer and interacts with the capture reagent in the capture layer, when present. In the case of the analyte-conjugation reagent complex, the complex binds to the capture reagent to provide a detectable, e.g., colorimetric signal. In essence, the capture reagent catches and accumulates the analyte-conjugation reagent complexes in the capture layer in order to provide the detectable signal. For the negative control, the analyte-conjugation reagent complex will still form, but it will not be captured by the capture layer because the capture antibody for the control zone in this layer is not present. For the positive control, an antibody specific to the metallic nanoparticle of the conjugation reagent is used.

The purpose of the absorbent layer is that it facilitates the rapid wicking of the sample to the test capture layer for colorimetric signal detection. The absorbent layer helps the solution to travel quickly to the capture layer for detection of the analyte.

The presence or absence of a signal in the detection region can be read by peeling back the layers above and/or below the detection layer to expose the signal. Once the test is complete, the layers on top of the capture layer is manually peeled off to expose the top side of the capture layer to check if a color signal is formed.

In an aspect, a method of detecting present or past SARS-CoV-2 infection, SARS-CoV-2 virus, and/or COVID-19 disease in a sample comprises
applying the sample to the sample application zone of the microfluidic device described herein,
optionally applying a buffer to the test sample to initiate flow and wash the layers, and
allowing the sample to flow from the sample layer, to the conjugation layer, to the capture layer, and
removing the layers that are on top of the capture layer to expose the capture zone of the capture layer,
wherein the presence of a detectable signal from the patient sample in the capture zone confirms a present or past SARS-CoV-2 infection, SARS-CoV-2 virus, and/or COVID-19 disease.

In an aspect, the sample comprises a patient sample such as a bodily fluid such as blood, blood plasma, serum, urine, saliva, milk, tear, sweat, nasal fluid (i.e., mucus), throat swab, nasal swab, any derivatives thereof, or a combination thereof.

In another aspect, the sample comprises an environmental sample, such as a sample collected from a surface, extracted from an item, drinking water, food, environmental fluid, wastewater, or a combination thereof. Samples may be collected from surfaces such as common items in public or private spaces such as door handles, computer keyboards in public places, desk/table surfaces, drinking water, food, wastewater, environmental fluid samples, and the like. Samples may be collected or extracted from hospital or lab items including personal protective equipment, lab coats, gloves, shield, masks to detect the presence of the SARS-CoV-2. It is important to test if the hospital items and the healthcare worker’s lab coats, gloves, shields, to find out if the items are contaminated or exposed to the virus.

In an aspect, the sample is applied in a volume of 10-100 µL. In another aspect, the analyte is detected in 5 minutes or less for singleplex and 10 minutes or less for multiplex.

Advantageously, the methods disclosed herein can be easily performed by a user having no medical experience and/or expertise. In the methods disclosed herein, strict procedures are not required to be followed, and samples, such as saliva, blood from finger pricking, or urine, do not need any pretreatment before testing. Additionally, the device is easily operated by non-trained medical personnel and has a light weight and small size (1.75 cm by 1.75 cm) for easy portability. By simply adding the required specimen type followed by a washing buffer and then peeling off the device readout layer, patients can readily interpret the colorimetric results of the test and determine if they are or have been infected by the virus using the naked eye. The possibility of patients being able to self-test from home further reduces the risk of spreading the virus.

In an aspect, the disclosed methods are conducted by a healthcare worker, a patient having and/or suspected of having present or past SARS-CoV-2 infection, SARS-CoV-2 virus, and/or COVID-19 disease, or any individual associated with the patient.

The invention is further illustrated by the following non-limiting examples.

### EXAMPLES

### Materials and Methods

Recombinant SARS-CoV-2 S1 [His], anti-SARS-CoV-2 Spike MAb (detection), anti-SARS-CoV-2 Spike MAb (capture), anti-SARS-CoV-2 Nucleocapsid MAb (detection), and anti-SARS-CoV-2 Nucleoprotein MAb (capture) were obtained from Creative Diagnostics (Shirley, NY). Rabbit IgG and Goat anti-Rabbit IgG were obtained from EMD Millipore-Sigma (Burlington, MA). Anti-Human IgG capture and anti-Human IgM capture Ab were obtained from Jackson ImmunoResearch Laboratories, Inc. (West Grove, PA). Pooled human nasal fluid was obtained from Innovative Research, Inc. (Novi, MI). Saliva and normal urine were obtained from Lee Biosolutions. Inc (Maryland Heights, MO). Whole human blood from healthy donors was acquired from Research Blood Components, LLC (Watertown, MA). Human Immunodeficiency Virus 1 (HIV 1) Antigen, Influenza A Virus Antigen, MERS-CoV Spike Protein S1, and SARS-CoV Spike Protein S1 were obtained from East Coast Biologics, Inc (North Berwick, ME). Bilirubin, cholesterol, human albumin, and human gamma globulin were obtained from MP Biomedicals, LLC (Solon, OH). Sucrose, uric acid, and Dulbecco’s phosphate buffer saline (DPBS) were acquired from Thermo Fisher Scientific, Inc. (Waltham, MA). Tween 20 was purchased from Sigma Aldrich (St. Louis, MO). Double-sided adhesive was provided by FLEXcon (Spencer. MA). The nitrocellulose membrane. Whatman® chromatography paper, and blotting paper were bought from GE Healthcare Life Sciences (Pittsburgh, PA).

The samples tested in the diagnostic platform were prepared with fresh human blood, saliva, nasal fluid, and urine spiked with SARS-CoV-2 spike and nucleocapsid proteins or antibodies against SARS-CoV-2 (IgM or IgG). SARS-CoV-2 positive blood samples in the presence of endogenous substances such as bilirubin, cholesterol, uric acid, albumin, and gamma globulin were prepared. Pretreatment of the specimens used in this study was not required prior to testing.

The statistical analyses were performed using GraphPad Prism (La Jolla. CA, U.S.A.). The statistical data was evaluated by one-way ANOVA in combination with Bonferroni tests. In this work, the data was represented as an average ± standard deviation (*p < 0.05, **p < 0.01. ***p < 0.001, and ****p < 0.0001)

### Example 1: Synthesis of Colloidal Gold-Anti-SARS-CoV-2 Proteins

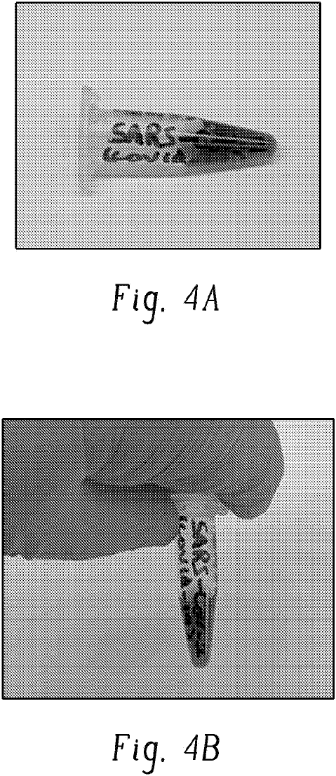

The point of care testing (POCT) platform was designed for singleplex detection of the spike or nucleocapsid antigenic proteins of SARS-CoV-2. In addition, multiplex detection of IgG/IgM antibodies against SARS-CoV-2-SP and spike antigenic protein was also performed. Therefore, the SARS-CoV-2 S-protein antigen, anti-spike protein mAb, and anti-Nucleocapsid protein mAb were conjugated to colloidal gold nanoparticles according to the manufacturer’s protocol (DCN Diagnostics, Carlsbad, CA). Briefly, each of the above-mentioned proteins were dialyzed in a 10 mM sodium phosphate buffer solution using 10 K MWCO dialysis devices. Then, conjugation was performed by mixing the colloidal gold solution (AuNP) titrated at the suggested pH values (7.0-9.0) with 0.004-0.012 mg/mL of each protein for 15 minutes at room temperature (RT). This was followed by blocking each solution for 30 minutes using the blocking solution in the conjugation kit. The resulting solutions were centrifuged at 14,000 x g for 10 minutes. The supernatant was removed, and the resulting conjugated anti-spike protein mAb, anti-nucleocapsid protein mAb, and S-protein antigen pellets were resuspended in the buffer provided by the kit. The resulting conjugated solutions were stored at 4° C. until use. An example of the gold-nanoparticle-conjugated anti-SARS-CoV-2 (N) protein antibody is shown in FIGS. 4A and 4B

### Example 2: Fabrication of the Paper-Based Diagnostic Platform for Singleplex and Multiplex Detection of SARS-CoV-2/COVID-19

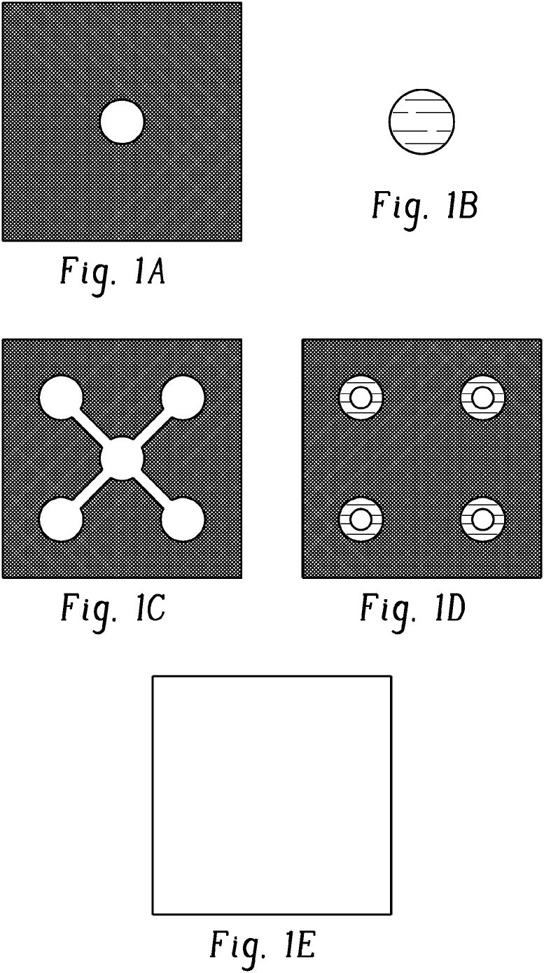

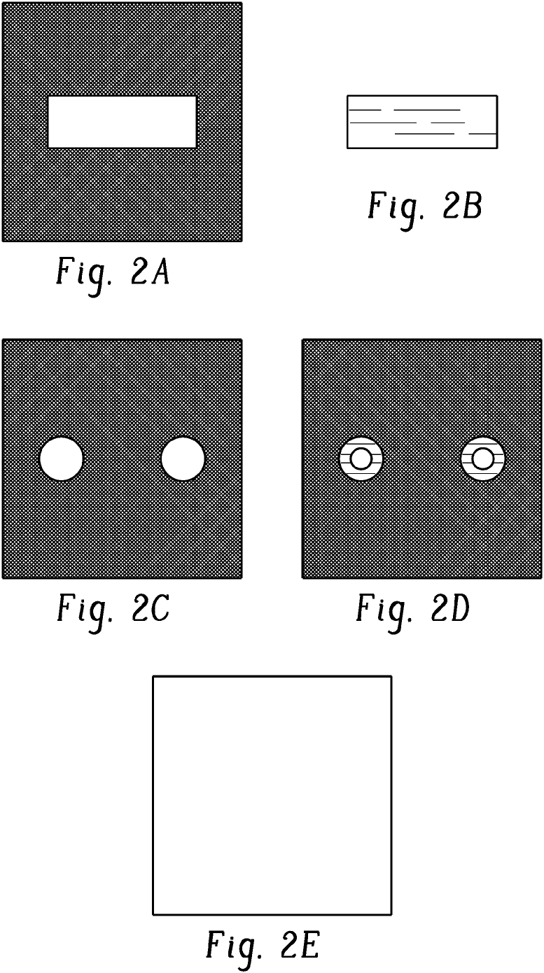

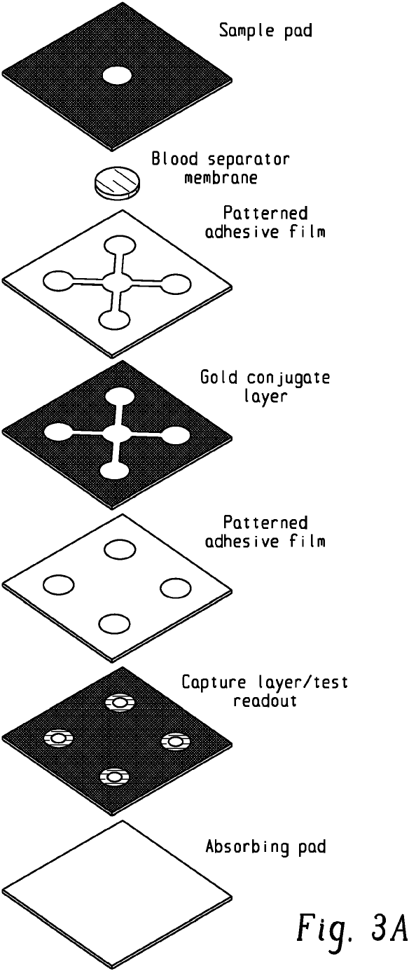

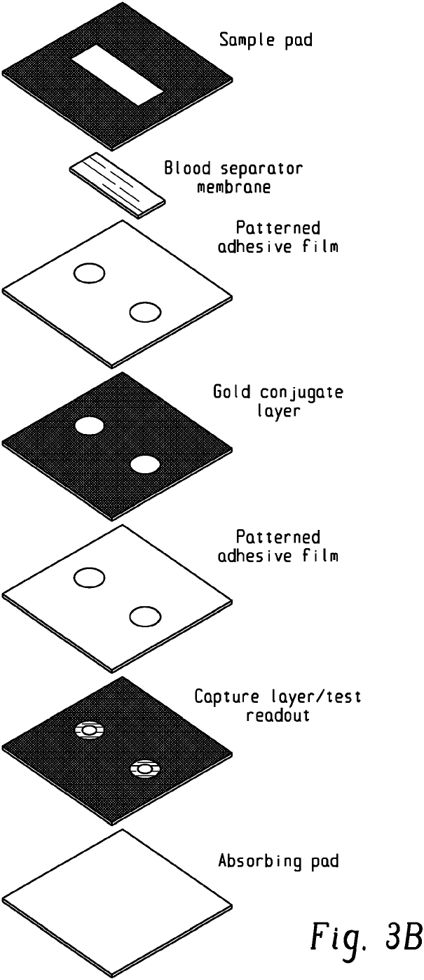

The 5-layer platform used in this study for singleplex or multiplex detection was designed using Adobe Illustrator (FIGS. 1A-1E and 2A-2E). The singleplex platform (FIGS. 2A-2E) was designed with only two testing zones on the device test readout layer. The testing zones enabled colorimetric detection of a single analyte and a control. Similarly, the multiplex platform (FIGS. 1A-1E) was designed with 4 testing zones for simultaneous detection of 3 analytes and a control. Wax printing using a Xerox ColorQube 8580 printer was used to create the hydrophobic areas that surround the active hydrophilic test zones on the proposed devices. This was done on every layer except the blotting and separator layers. The sample pad and conjugate layers of these devices were printed on Whatman® No 1 chromatography sheets. The test readout layer was printed on the nitrocellulose membrane. To induce the melting of the wax, which created hydrophobic boundaries to define the sample zones, the paper layers were baked in an oven at 130° C. for 30 seconds. Ultimately, the devices were cut using a guillotine-type paper cutter, and the layers of the devices were assembled. (FIGS. 3A-3B) To assemble the layers of the device together, adhesive films patterned with open holes and channels were created with a laser cutter (GuangZhou Amonstar Trade CO., Ltd, KH-3020) and placed on the backside of each layer were placed on the back-side of each layer prior to assembling. The geometrical design of each layer of the adhesive film was produced using the Adobe Illustrator software. The adhesive film functions as a double-sided tape and sticks to the layers above and underneath the adhesive film itself. Lastly, the device was created by stacking all the layers one after the other, starting with the sample pad as the first layer and the blotting paper as the final bottom layer (FIGS. 3A-3B), to create a stacked ELISA-based assay. The fully assembled device dimensions were the same (1.75 cm by 1.75 cm) for the singleplex and multiplex platforms..

### Example 3: Immunoassay Implementation of Singleplex and Multiplex Testing Platform

Preparation of the immunoassay platform for singleplex detection (SARS-CoV-2-NP or SP) included the treatment of the conjugate layer with a conjugate treatment solution (0.05% Tween 20 and 1% BSA). The conjugate layer was then treated with a colloidal gold-labeled anti-SARS-CoV-2-NP or SP antibody solution. The solution was allowed to air dry at room temperature. This was followed by treating the test readout layer with the anti-SARS-CoV-2-NP or SP capture antibodies at 3 and 4 mg/mL concentrations, respectively. The test layer was then treated with a blocking solution (0.05% Tween 20 and 1% BSA). Next, the layers of these devices were assembled as explained in section 2.4. Testing was then initiated by adding 15 µL of samples positive to the SARS-CoV-2-NP or SP (100 µg/mL, 100 ng/mL, or 100 pg/mL) to the device sample zone. This was followed by the immediate addition of 30 µL of washing buffer (DPBS). The results were interpreted by peeling the devices’ layers apart to reveal the colorimetric results in test readout layer. Color interpretation was possible using the naked eye and quantified on images taken by an Android phone using the NIH ImageJ software. The grey intensity was quantified, and statistical analysis of the results was carried out. The detection time was within 5 minutes, and triplicate tests were performed for each concentration tested. Similarly, the same procedure was followed for the multiplex immunoassay platform preparation. Briefly, the conjugate layer was treated with colloidal gold-labeled anti-SARS-CoV-2-SP antibody, SARS-CoV-2 S-protein antigen (IgG/IgM), and Rabbit IgG (positive control) at the corresponding test zones. In addition, the test readout layer was treated with a 2 mg/mL antibody solution of anti-SARS-CoV-2 S-protein, anti-Human IgG/IgM. or anti-Rabbit IgG. Devices were assembled, and testing was performed in triplicates at 100 µg/mL in unmodified human blood. Testing took 10 minutes.

### Example 4: Analytical Specificity for Pathogenic Viruses and Endogenous Interferents

The cross-reactivity and possible interference of the singleplex platform for SARS-CoV-2 SP detection was also assessed by testing antigens of different pathogenic viruses (SARS-CoV, MERS-CoV, HIV, and Influenza A) and endogenous substances that can potentially interfere with the detection efficiency of this testing platform. Each pathogenic virus was analyzed at a concentration of 100 ng/mL in samples negative for SARS-CoV-2. The diagnostic platform was challenged with individual blood samples positive for each virus at the concentration specified above. The specificity of the platform was evaluated by quantifying the red color signal observed in the test zone of each device as a result of the detection of each pathogenic virus. Statistical analysis was performed, and the results were compared to a negative control and the positive signal for SARS-CoV-2 detection. Interference for SARS-CoV-2 detection in the presence of endogenous substances was also evaluated in the diagnostic platform. A cocktail composed of 10 ng/mL SARS-CoV-2 SP viral antigen, 0.2 mg/mL uric acid, 120 mg/mL albumin. 120 mg/mL gamma globulin. 0.6 mg/mL bilirubin, and 5 mg/mL cholesterol in unmodified human blood was tested. Samples were tested in replicates of three, and colorimetric results were evaluated by quantification using NIH ImageJ software. Statistical analysis was performed to determine if the presence of potentially interferent substances altered the ability of the platform to detect SARS-CoV-2.

### Example 5: SARS-CoV-2 Detection Performance in the Presence of Different Bodily Fluids

To examine the detection profile of the proposed POCT platform in different bodily fluids, specimens such as saliva, unmodified human blood, nasal fluid, and urine were spiked with SARS-CoV-2 antigenic S-protein at a concentration of 100 ng/mL. The specimens were then individually tested in the singleplex POCT platform by adding 15 µL of the specimen to the device sample zone. Colorimetric results were observed within 5 minutes, and the results were quantified using NIH ImageJ software. Statistical analysis was used to evaluate differences in the detection sensitivity of the assay as a result of changes in the sample media.

### Results and Discussion

A rapid and tunable platform for SARS-CoV-2/COVID-19 diagnostics was developed to provide a simple, flexible, low-cost, portable, and immunoassay-based vertical flow platform that performs rapid point-of-care diagnosis of COVID-19 at all stages of the disease development. Singleplex testing of the SARS-CoV-2 virus spike protein (SP) and nucleocapsid protein (NP) were evaluated. In addition, multiplex detection of SARS-CoV-2 antibodies (IgG and IgM) and SARS-CoV-2 antigens in a single device was examined. Furthermore, the specificity of the platform for the detection of the SARS-CoV-2-SP in the presence of other cross-reactant viruses and endogenous substances was characterized. The detection of the SARS-CoV-2-SP in different bodily fluids was also studied.

### Platform Development for SARS-CoV-2/COVID-19 Detection

Indication of a COVID-19 infection is extremely nonspecific since the symptoms of this disease are similar to those of other illnesses such as viral pneumonia, cough, or fever. Therefore, user-friendly, rapid, and low-cost point-of-care diagnostic platforms with high specificity are critically needed for confirming suspected COVID-19 cases and limiting the spread of the virus among communities. See Nguyen et al, Micromachines (Basel), 2020, 11(3): p. 306. Currently, COVID-19 diagnostics are limited to a specific type of test, sample type, or stage of the disease. These key factors affect the test outcomes of current COVID-19 diagnostic tools potentially leading to misdiagnosis or false results. See Toptan et al, J. Clin. Virol. 2021, 135: p. 104713; Hu et al, Preprints 2020, 2020040155 (doi: 10.20944/preprints202004.0155.v1).

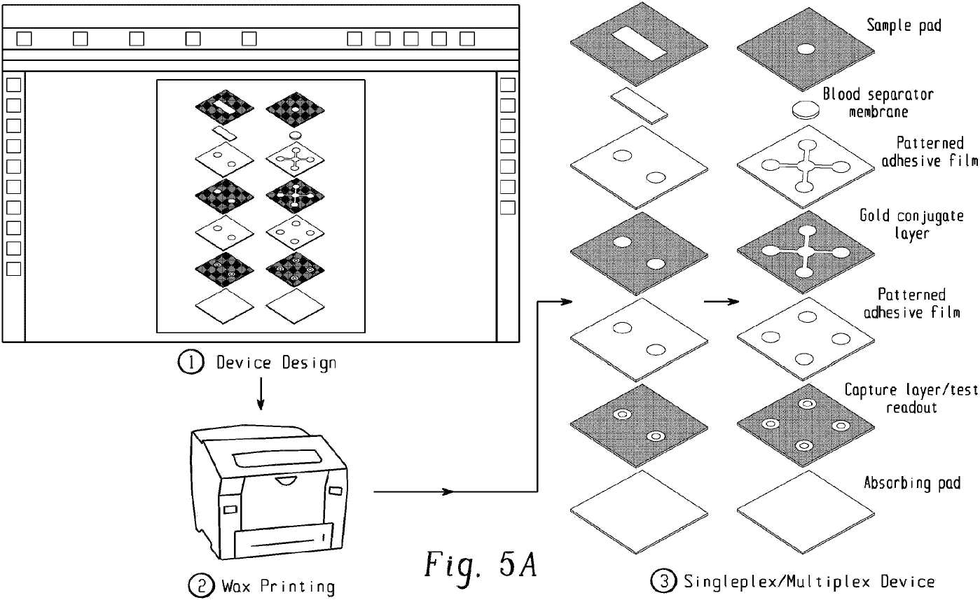

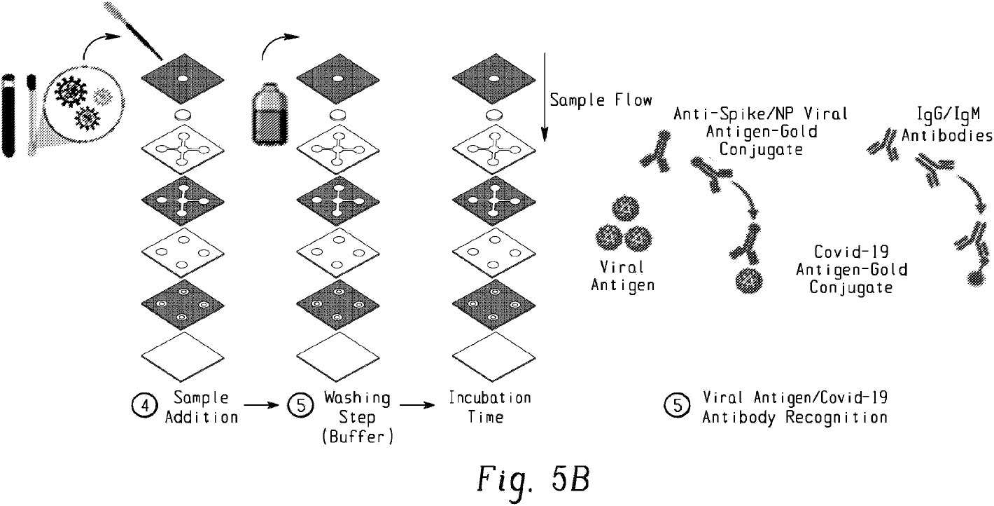

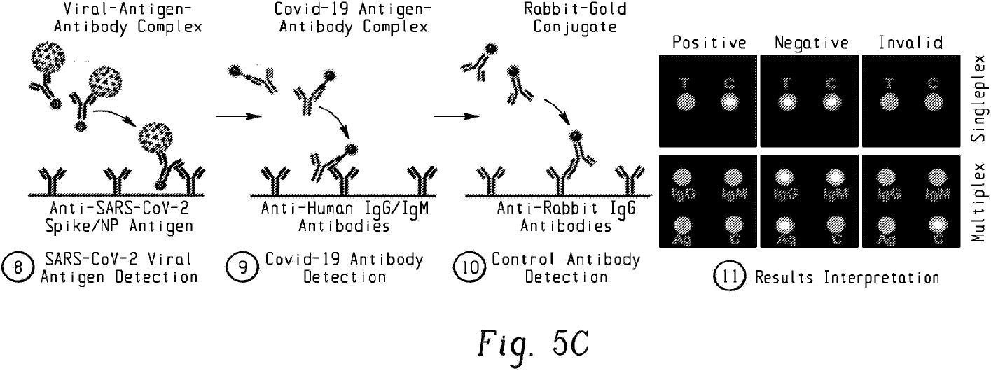

In this work, a flexible paper-based platform for singleplex and multiplex colorimetric detection of SARS-CoV-2/COVID-19 was developed by integrating the principles of sandwich enzyme-linked immunosorbent assay (ELISA) with microfluidics in a vertical flow arrangement (FIGS. 5). The singleplex platform was engineered for rapid individual testing of single antigenic proteins of the SARS-CoV-2 virus (SP or NP) in one diagnostic device (FIGS. 2A-2E). Detection of multiple analytes (SARS-CoV-2 (SP) and IgG/IgM antibodies) was also accomplished in a single diagnostic device with the multiplex detection platform (FIGS. 1A-1E). A vertical flow microfluidics approach was utilized during the development of the 5-layer single-plex and multiplex platforms because it was ideal for the rapid diagnosis of SARS-CoV-2/COVID-19 within sample volumes and time frames appropriate for minimally invasive POC diagnostics (e.g.. <50 µL and <10 minutes). This approach allowed for rapid detection performance and flexibility in the multiplex diagnostic capability of the platform. Additionally, it eliminated hook effect problems, which are limiting factors in current diagnostic devices where lateral flow microfluidics is utilized as a proposed method for sample wicking to the device test zone. See Nguyen et al, supra; Hu et al, supra; Moumita et al, Food Chemistry. 19 Jul. 2018, 270:585-592. In addition, literature studies have shown vertical flow microfluids to be up to 5 times more sensitive than lateral flow approaches. See Nguyen et al, supra. Therefore, a vertical flow approach was the selected method for increasing the sensitivity and limiting the possibility of false negative results, especially in the later stages of COVID-19 disease development where the viral load may be reduced.

Throughout the development of this platform, key factors such as ease of use in operating the test and evaluating the final results, portability, and specimen type were considered. This diagnostic platform was developed for users with no medical experience. As such, strict procedures are not required to be followed, and samples, such as saliva, blood from finger pricking, or urine, will not need any pretreatment before testing. Literature studies have demonstrated that saliva is a reliable specimen for SARS-CoV-2 detection because it causes less patient discomfort and is a useful non-invasive sample for patient self-collection. See Williams et al. J. Clin. Microbiol., 2020, Jul 23; 58(8): e00776-20; Wyllie et al. N. Engl. J. Med.. 2020. 383(13): p. 1283-1286; To et al, Lancet Infect. Dis., 2020, 20(5): p. 565-574. In addition, whole blood specimens obtained by finger pricking are desired for SARS-CoV-2 antibody testing because they eliminate major pre-analytical obstacles such as pre-treatment of blood specimens (centrifugation). Furthermore, venous blood sampling prevents patients from self-testing at point of care. See Andrey, et al., Eur. J. Clin. Invest.. 2020. 50(10): p. e13357. Therefore, the flexibility, simplicity, and ability of this platform to use different specimen types without pre-treatment procedures were important parts in the successful development of the immunoassay platform. Additionally, the ability of the platform to be easily operated by non-trained medical personnel and its light weight and small size (1.75 cm by 1.75 cm) for easy portability were important factors also considered and optimized. Simple operation and portability of POCT devices are key requirements for the effective utility of POCT devices in various testing settings. See Omidfar, et al., Biosens. Bioelectron,, 2013, 43: p. 336-347. By simply adding the required specimen type followed by a washing buffer and then peeling off the device readout layer, patients can interpret the colorimetric results of the test and determine if they infected by the virus using the naked eye. The possibility of patients being able to self-test from home further reduces the risk of spreading the virus.

The sensitivity, specificity, and colorimetric detection efficacy of this immunoassay platform were other key factors considered during the development process of this platform. To elaborate, optimal colorimetric results and higher sensitivities during analyte testing were obtained by using 20 nm gold nanoparticles during the gold nanoparticle-conjugation process of monoclonal antibodies (mAbs) against SARS-CoV-2 antigenic proteins (SP and NP) and anti-SARS-CoV-2-SP antibodies in the singleplex and multiplex platforms, respectively. It is well established in literature studies that gold nanoparticles of approximately 20 nm in size are preferred for fabrication of rapid immunoassays because they provide optimal colorimetric results at high intensities visible to the naked eye and enhance the detection sensitivity of the assay platform. See Omdifar at al., supra; Lou, et al., Analyst, 2012, 137(5): p. 1174-1181. In addition, literature examples have also specified the need for specific antibodies against SARS-CoV-2 to increase the specificity of current immunoassays and eliminate cross reactivity problems that may result from considerable homology between SARS-CoV-2 and other coronaviruses, such as SARS-CoV. See Terry, J.S., et al., Virology, 2021. 558: p. 28-37. Thus, commercially available monoclonal antibodies specific for SARS-CoV-2 detection were utilized in this platform.

Overall, the engineering approach used to develop this paper-based diagnostic platform allowed for singleplex detection of the antigenic proteins (SP and NP) for the SARS-CoV-2 virus in various specimen types without pre-treatment prior to testing. Only 15 µL of the testing analyte were required, and results were obtained in 5 minutes. In addition, the flexibility of this platform for multiplexing also allowed for rapid detection of multiple SARS-CoV-2/COVID-19 analytes (SARS- SARS-CoV-2-S(P) and IgG/IgM antibodies) in 10 minutes using only 30 µL of unmodified human blood in a single device. Altogether, the novel platform’s ability to be tailored to a specific detection pattern and sample type, its portability, and its low cost, as a result of inexpensive paper-based materials used for its fabrication, demonstrate the ability of this platform to be a potential alternative for rapid testing of SARS-CoV-2/COVID-19 in various testing sites.

### SARS-CoV-2/COVID-19 Platform Detection Capability

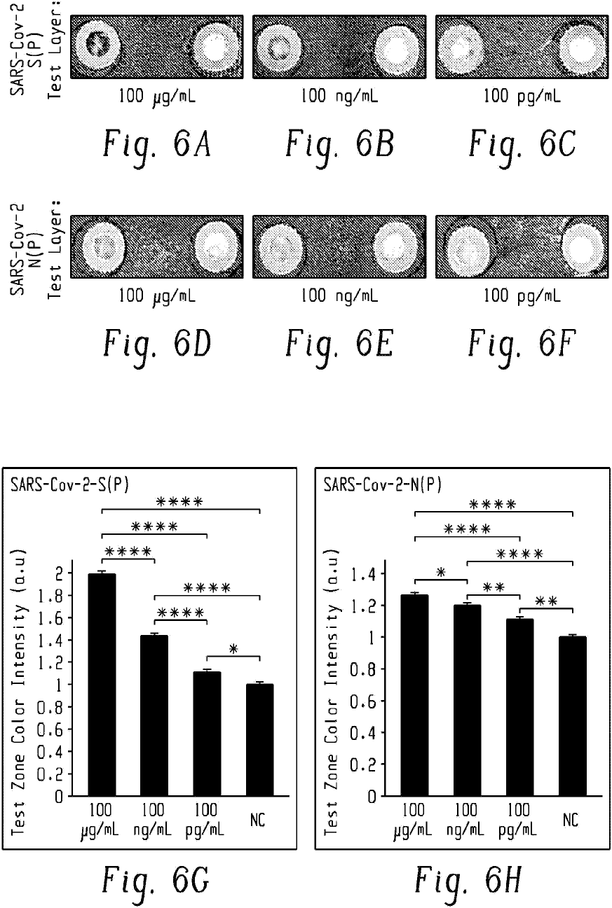

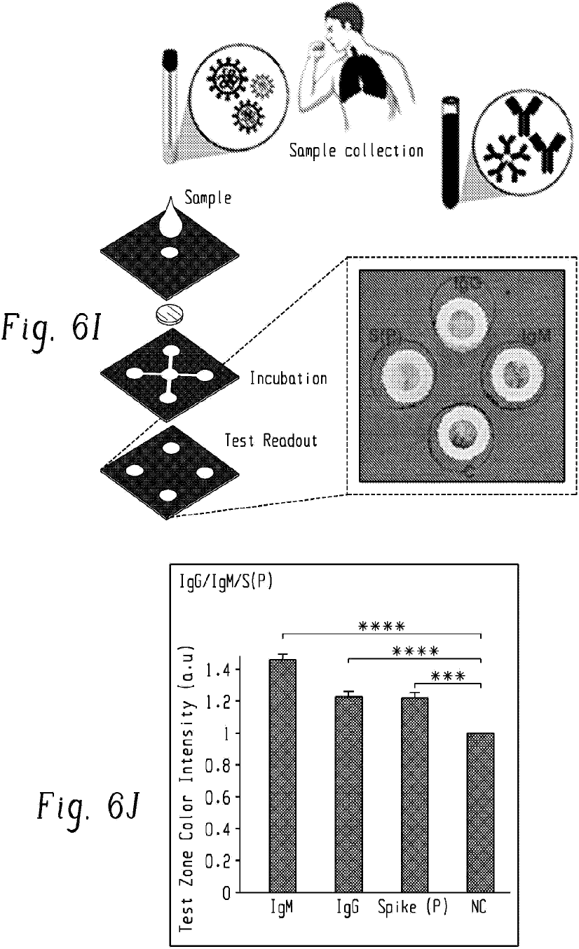

The detection capability of the flexible POCT platform for SARS-CoV-2/COVID-19 diagnosis was confirmed by performing testing of SARS-CoV-2 antigenic species (SP and NP) and antibodies (IgG/IgM) against the SARS-CoV-2-S(P) virus in unmodified fresh human blood. To demonstrate the detection affinity of the singleplex platform to SARS-CoV-2 SP and NP viral antigens alone, diluted samples at concentrations ranging from 100 µg/mL to 100 pg/mL were added to the sample zones on the devices as shown in FIGS. 6A-6F. Colorimetric results were evaluated within 5 minutes after the sample addition by quantifying the color intensity formed in the device’s test zone using images taken with an Android cellphone. NIH ImageJ software was used for quantification of the colorimetric results, and significant differences among detected concentrations were assessed through statistical analysis (FIGS. 6G-6H). In FIGS. 6A-6H, the colorimetric results for the detection of the SARS-CoV-2 SP and NP antigenic species showed a decreasing trend in color intensity as the concentration of the SP and NP viral antigens in the tested samples decreased, as expected. However, the detection affinity of the mAbs used in this sandwich immunoassay against the SARS-CoV-2 SP viral antigen proved to be higher compared to the results shown for the detection of the SARS-CoV-2 NP antigen. These results are in accordance with literature studies showing higher sensitivities for singleplex assays targeting both of these antigenic species individually in single devices. See Cai, et al., Anal. Bioanal. Chem., 2021, May 31, p. 1-10. The results for the detection sensitivity of the SARS-CoV-2 SP antigen is superior, and limits of detection (LOD) for each detected viral analyte (SP and NP) are within similar concentration ranges as tested in this assay (100 pg/mL). See Cai, supra. However, given the ability of the diagnostic platform to be flexible and meet specific testing requirements, this platform can potentially be arranged for multiplex detection of both antigenic species in a single device and contribute to more valid testing results if necessary. See Barlev-Gross, et al., Anal. Bioanal. Chem., 2021. 413(13): p. 3501-3510. In FIGS. 6G-6H, the quantification results for the detection of both SARS-CoV-2 antigenic proteins confirm the colorimetric observations presented in this assay. In addition, significant statistical differences between each concentration (100 µg/mL, 100 ng/mL, and 100 pg/mL) tested for each antigenic protein were observed. Similarly, a significant statistical difference was also observed between each concentration and the negative control test zone in the assay.

The multiplexing capability of this diagnostic platform was also assessed through preliminary studies where the SARS-CoV-2 SP viral subunit and IgG/IgM antibodies against SARS-CoV-2-S(P) were targeted simultaneously within one diagnostic device (FIGS. 6I-6J). This study was performed as a proof-of-principle to demonstrate the flexibility of this diagnostic platform and its ability to be easily customized for multiplexed testing for COVID-19. Multiplexing diagnostic tests for COVID-19 has the potential to accelerate the detection of COVID-19 at different stages of the disease or infection progress. In addition, such diagnostic tools could be valuable in sites (e.g. school) where information regarding immunity and infection could be beneficial for proper quarantine measures. See Masterson, et al., Anal. Chem., 2021, 93, 25, p. 8754-8763. To perform this experiment, a sample spiked with each targeted analyte in unmodified human blood at a 100 µg/mL concentration was used. As shown in the results in FIG. 6I a colorimetric signal for the analytes tested in this platform was observed in all the individual test zones specific for each analyte (IgG, IgM, SP viral antigen, and positive control labeled as C). To confirm the absence of non-specific binding due to false-positive results, previous experiments using solutions negative to each analyte were also performed, and colorimetric results were only observed in the positive control test zone, as expected. These results suggest no interference between the detection capability of each detection and capture mAb utilized for sandwich ELISA in this platform. Additionally, the quantified results in FIG. 6J demonstrated statistically significant differences when colorimetric results from each targeted analyte were compared to a negative control.

The overall results of this study validated the ability of the diagnostic platform to perform rapid detection of SARS-CoV-2 viral subunits at concentrations as low as 100 pg/mL and serological testing against SARS-CoV-2 antibodies. The colorimetric results can be qualitatively assessed by the naked eye. Three replicates were performed for each test, and no false positive results were observed during this work.

### Analytical Specificity of COVID-19 Platform for SARS-CoV-2 Antigen Detection in the Presence of Other Viruses and Potential Interferents

In this work, the cross-reactivity and possible interference of the singleplex platform for SARS-CoV-2 SP detection was also assessed by testing different pathogenic viruses (SARS-CoV, MERS-CoV, HIV, and Influenza A) and endogenous substances that can potentially interfere with the detection efficiency of the platform. The spike antigens of each pathogenic virus were analyzed at a 100 ng/mL concentration in samples negative to SARS-CoV-2.

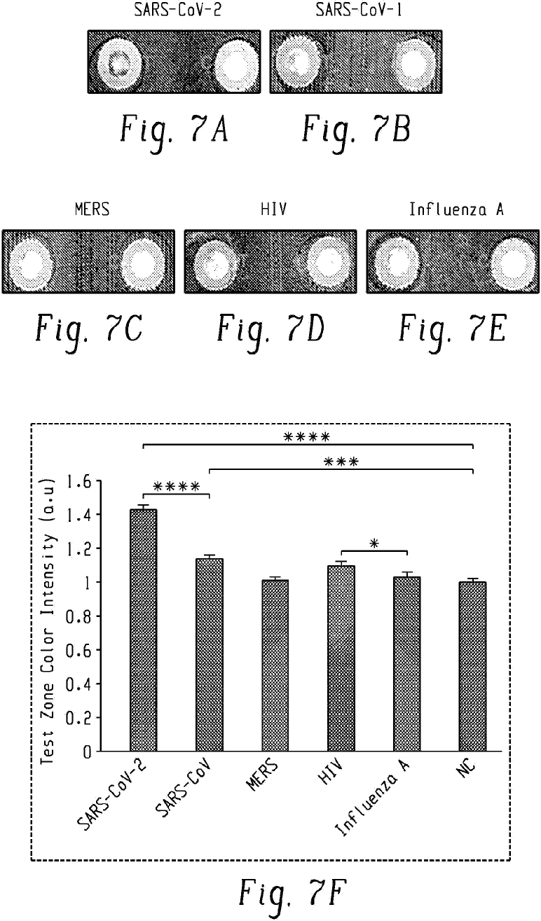

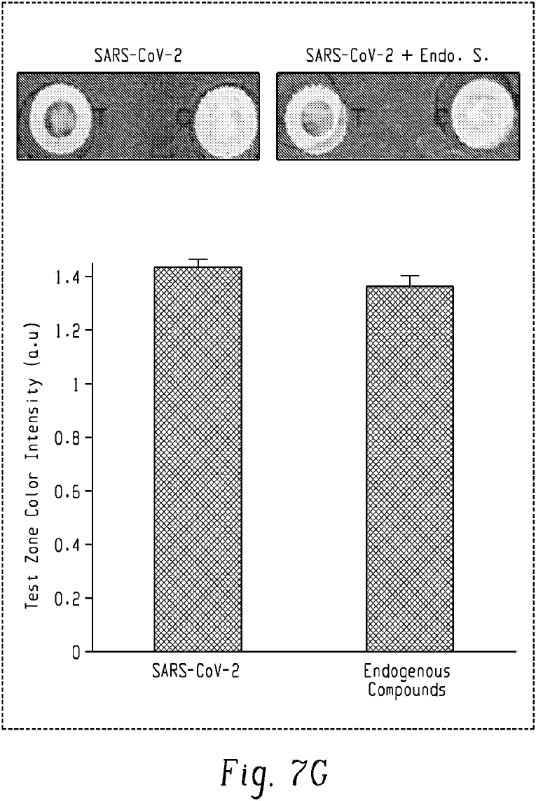

Interferent endogenous substances were tested in the presence of the SARS-CoV-2 SP (100 ng/mL) in unmodified human blood. Samples were tested in replicates of three, and colorimetric results were evaluated using NIH ImageJ software to quantify color intensity (FIGS. 7A-7G). The tests performed in this study were conducted following the current recommendations issued by the FDA for data submission requests to support the pre-EUA/EUA of a diagnostic POCT for SARS-CoV-2 antigen testing. See FDA, Coronavirus Disease 2019 (COVID-19) Emergency Use Authorizations for Medical Devices, M. Devices, Editor.

SARS-CoV showed only a slight red color signal in the testing zone of the POCT platform when challenged with a blood sample positive for SARS-CoV (100 ng/mL). The detection of MERS showed no red color formation when tested at the same 100 ng/mL concentration. This may be due to SARS-CoV sharing similar homology with SARS-CoV-2. See Terry, supra. However, FIG. 7F demonstrates a significant difference in the signal intensity for the quantification result for SARS-CoV-2 when detected at the same concentration (100 ng/mL) and compared to the results obtained for SARS-CoV. The colorimetric detection signal of SARS-CoV-2 is evidently in the higher end of the LOD of this diagnostic platform. The signal intensity resulting from the quantification result for MERS is confirmed to be not statistically significant because it is in the same quantification range as the negative control (NC, 0 ng/mL). Therefore, these results suggest the possibility of this diagnostic platform to distinguish among different coronaviruses since these results can only be expected from POCT tools with enhanced specificity against SARS-CoV-2, which seems to be the case for the detection platform. A similar testing trend has also been observed in other literature examples, but colorimetric differentiation with the naked eye was less evident. See Lee, et al., Biosens. Bioelectron., 2021, 171: p. 112715. Furthermore, the quantification results for Influenza A showed no cross reactivity as its quantification results were similar to the negative control (NC). The quantification results for the HIV virus, however, seemed to show a minimal cross-reaction during detection. Lastly, in FIG. 7G the results for SARS-CoV-2 detection (10 ng/mL) in the presence of various endogenous compounds (0.6 mg/mL bilirubin, 5 mg/mL cholesterol, 0.2 mg/mL uric acid, 120 mg/mL albumin, and 120 mg/mL gamma globulin) showed no significant statistical difference when compared to the device tested with a solution in the presence of SARS-CoV-2 only. Overall, the COVID-19 platform presented in this study demonstrates enhanced sensitivity in detecting the SARS-CoV-2 SP antigen with minimum cross reactivity problems as a result of interference with other organisms. In addition, this platform proved to be potentially suitable to differentiate among coronaviruses.

### SARS-CoV-2 Detection Performance in the Presence of Different Bodily Fluids

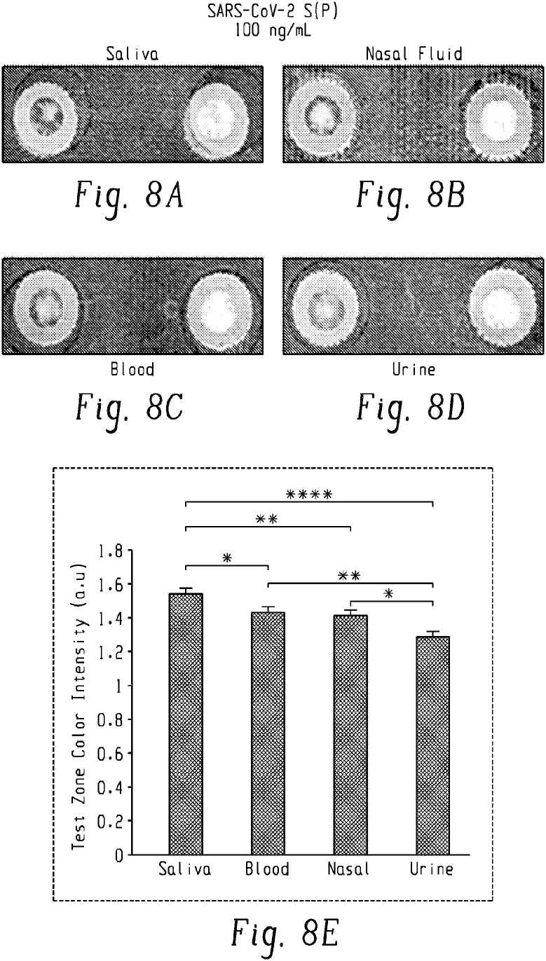

Appropriate specimen type and sample collection are key factors for determining the successful performance of SARS-CoV-2 diagnostic tools. However, depending on the application and method for diagnosis, a suitable sample type must be used. Among them, nasal fluid, as well as saliva, urine, and blood, have been used to diagnose patients infected by the COVID-19 virus. To further investigate the detection profile of the singleplex immunoassay platform, various specimen types (saliva, nasal fluid, blood, and urine) were tested (FIGS. 8A-8E). Each specimen was spiked with the SARS-CoV-2 SP at 100 ng/mL, and statistical analysis was performed to evaluate the results. Triplicate experiments were done for each specimen type.

In FIGS. 8A-8D, colorimetric results can be observed in the test readout layer for each diagnostic device where a different specimen type was tested. Non-false positive results were observed, and color intensity on the sample test zone could be differentiated from the NC zone in the tested devices by the naked eye. These results suggest the potential capability of this diagnostic tool to be adapted to work with multiple specimen types if they are appropriate for the detection method of a targeted SARS-CoV-2 analyte. Interestingly, the quantification results in FIG. 8E demonstrate that the saliva sample containing the SARS-CoV-2 SP analyte provided the highest signal intensity (sensitivity) among all tested specimens. Blood samples showed the second highest sensitivity followed by nasal fluid. A statistical difference was observed between the detection signal of all specimen types, except no significant difference was observed between blood and nasal fluid. The urine specimen showed the lowest sensitivity among all samples. These results are expected as literature studies have not shown a lot of evidence supporting the presence of viral ribonucleic acid (RNA) in urine. See Peng, et al., J. Med. Virol., 2020, 92(9): p. 1676-1680. However, several literature studies have suggested that SARS-CoV-2 detection in saliva samples provides results with higher sensitivity than results obtained using nasopharyngeal specimens. See Williams, at al, supra; To, et al supra; Kashiwagi, et al., J. Infect. Chemother., 2021, 27(2): p. 384-386; Wyllie, at al, supra. Since saliva is a reliable and non-invasive sample type for patient self-collection, the ability of this platform to provide the highest sensitivity results within this specimen type serves as steppingstone for advancing this research closer to a platform for SARS-CoV-2 self-testing at home.

Overall, the ability of this platform to provide successful results by allowing for the testing of various specimen types without pre-treatment in the same platform may be a valuable solution for establishing a better prognosis for patients infected by the SARS-CoV-2 virus.

A flexible paper-based platform for singleplex and multiplex colorimetric detection of SARS-CoV-2/COVID-19 has been developed based on the combination of vertical flow microfluidics and sandwich immunoassay. This approach allowed for high assay sensitivity and rapid results within time frames and specimen volumes closer to minimally invasive diagnostics (e.g.. <50 µL and <10 minutes). The test results can be interpreted using the naked eye by untrained users similar to home-based pregnancy tests. Multiple specimen types can be used to detect SARS-CoV-2 related analytes in the testing platform. The various experiments indicated that the rapid SARS-CoV-2 detection assay device allowed for the efficient and effective wicking of the targeted samples through the designated sample test zones in the device. The singleplex design of the platform allowed for individual testing of the spike and nucleocapsid antigenic proteins of the SARS-CoV-2 virus at concentrations within the 100 pg/mL detection range. In particular, the device successfully detected microgram, nanogram, and picogram concentrations of SARS-CoV-2 antigen in human blood and produced a clear readout within a few minutes. Additionally, the multiplex detection design of the POC device demonstrated simultaneous detection of IgG and IgM antibodies and the SARS-CoV-2 SP in a single platform using human blood. The assay platform provided non-significant interference against other pathogenic viruses and demonstrated the potential to differentiate between other coronaviruses such as MERS-CoV and SARS-CoV. To further asses the detection profile of this diagnostic platform, singleplex testing was performed using various specimen types. While the platform achieved SARS-CoV-2 detection in blood, nasal fluid, saliva, and urine, it was found that saliva provided the most sensitive results. The technology thus offers not only a potential rapid and low-cost alternative to current immunoassay-based techniques for SARS-CoV-2 diagnostics, but also provides flexibility by being able to adapt to the specific testing requirements needed by various testing locations.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A multilayer diagnostic microfluidic device for the capture of a present or past SARS-CoV-2 infection and/or COVID-19 disease, the device comprising a sample application layer comprising a sample application zone, a first side of a conjugation layer comprising a conjugation reagent in a conjugation zone, wherein the conjugation reagent comprises a detectable marker, a capture layer comprising a capture reagent in a capture zone, and an absorbent layer, wherein the sample application layer is in communication with a first side of the conjugation layer, a second side of the conjugation layer is in communication with a first side of the capture layer, and the second side of the capture layer is in communication with a first side of the absorbent layer, wherein the sample application layer, the conjugation layer, the capture layer, and the absorbent layer each comprise a porous media, wherein the sample application zone, conjugation zone, and the capture zone are in communication such that an analyte in the sample application zone binds to the conjugation reagent in the conjugation reagent zone to form an analyte- conjugation reagent complex, and the analyte-conjugation reagent complex binds to the capture reagent in the capture zone to form a detectable signal in the capture zone, wherein the sample application layer and the conjugation layer are above the capture layer and are removable to expose the detectable signal in the capture zone, and wherein the analyte is a SARS-CoV-2 protein or an antigenic fragment thereof, an IgM, IgG, or IgA antibody associated with a SARS-CoV-2 infection and/or COVID-19 disease, or an inflammatory biomarker associated with a SARS-CoV-2 infection and/or COVID-19 disease, and wherein the detectable signal in the capture zone indicates a present or past SARS-CoV- 2 infection and/or COVID-19 disease.

2. The multilayer microfluidic device of claim 1, further comprising a sample treatment layer disposed on a side of the sample application layer opposite the conjugation layer, wherein the sample treatment layer comprises porous media.

3. The multilayer microfluidic device of claim 2, wherein the sample treatment layer filters the sample as it enters the device.

4. The multilayer microfluidic device of claim 2, wherein the sample treatment layer separates plasma from whole blood.

5. The multilayer microfluidic device of claim 1-, wherein the device is a singleplex or multiplex device comprising a plurality of sample application zones in the sample application layer, wherein the plurality of sample application zones are in communication with a plurality of conjugation reagent zones in the conjugation layer, wherein the plurality of conjugation reagent zones are in communication with a plurality of capture zones in the capture layer, and wherein each of the plurality of conjugation reagent zones comprises the same or a different conjugation reagent.

6. The multilayer microfluidic device of claim 1, comprising between one and ten sample application zones, between two and ten conjugation zones, and between two and ten capture zones.

7. The multilayer microfluidic device of claim 1, wherein the multiplex device comprises a conjugation reagent for conjugation of at least one SARS-CoV-2 protein or an antigenic fragment thereof, at least one IgM, IgA or IgG antibody associated with a SARS-CoV-2 infection and/or COVID-19 disease, and optionally at least one inflammatory biomarker associated with a SARS-CoV-2 infection and/or COVID-19 disease.

8. The multilayer microfluidic device of claim 1, wherein the analyte is a subunit, isoform, or antigenic peptide of SARS-CoV-2 Spike (S) protein, SARS-CoV-2 Membrane (M) protein, SARS-CoV-2 Nucleocapsid (N) protein, SARS-CoV-2 Envelope (E) protein, or a combination thereof.

9. The multilayer microfluidic device of claim 1, wherein the sample application layer, the conjugation layer and the capture layer each comprise a hydrophobic material that defines the boundary of the sample application zone, the conjugation zone, and the capture zone, respectively.

10. The multilayer microfluidic device of claim 1, wherein the device further comprises one or more adhesive layers disposed between the sample layer and the conjugation layer, and/or, between the conjugation layer and the capture layer.

11. The multilayer microfluidic device of claim 1, further comprising a control zone, wherein the portion of the capture layer associated with the control zone comprises no capture reagent, a capture reagent against the gold-nanoparticle labeled antibody, or a combination thereof.

12. The multilayer microfluidic device of claim 1-, wherein picogram to microgram amounts of the analyte are detectable, specifically wherein picogram to nanogram amounts of analyte are detectable.

13. The multilayer microfluidic device of claim 1, wherein the detectable signal is a colorimetric signal.

14. The multilayer microfluidic device of claim 1, wherein the conjugation and capture reagents comprise an antibody or antibody fragment, protein, biomolecule, or an antigen.

15. The multilayer microfluidic device of claim 1, wherein the conjugation reagent comprises an antibody-metallic nanoparticle conjugate, such as a gold nanoparticle.

16. The multilayer microfluidic device of claim 1, wherein the porous media comprises paper, fabric, cotton, nylon, or plastic.

17. A method of detecting a present or past SARS-CoV-2 infection, SARS-CoV-2 virus, and/or COVID-19 disease in a sample, the method comprising applying the sample to the sample application zone of the microfluidic device of any of the foregoing claims, optionally applying a buffer to the test sample to initiate flow and wash the layers, and allowing the sample to flow from the sample layer, to the conjugation layer, and to the capture layer, and removing the layers that are on top of the capture layer to expose the capture zone of the capture layer, wherein the presence of a detectable signal from the patient sample in the capture zone confirms a present or past SARS-CoV-2 infection and/or COVID-19 disease.

18. The method of claim 17, wherein the sample comprises a patient sample comprising blood, blood plasma, serum, urine, saliva, milk, tear, sweat, nasal fluid, throat swab, nasal swab, a derivative thereof, or a combination thereof.

19. The method of claim 17, wherein the sample comprises an environmental sample comprising a sample collected from a surface, extracted from an item, drinking water, food, wastewater, or a combination thereof.

20. The method of claim 19, wherein the sample is applied in a volume of 10 µL to 100 µL and wherein the analyte is detected in less than 5 minutes in singleplex and 10 minutes for multiplexed devices.

21. (canceled)
